(19) United States
(12) Patent Application Publication
(10) Pub. No.: US 2009/0270976 A1
(43) Pub. Date: Oct. 29, 2009

(54) Stent Graft Fixation System and Method of Use

(75) Inventors: Nareak Douk, Lowell, MA (US); Nasser Rafiee, Andover, MA (US); Morgan House, Newfields, NH (US); Walter Bruszewski, Guerneville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/109,177

(22) Filed: Apr. 24, 2008

(51) Int. Cl. A61F 2/06

(52) U.S. Cl. 623/1.36; 623/1.13; 623/1.11

(57) ABSTRACT

A stent graft fixation system and method of use includes a stent graft system for fixation to a vessel wall, the system having a helical anchor and a stent graft. The helical anchor has a number of coils with a point at one end and a helical anchor axis, and the stent graft has a stent graft axis. The coils are operable to sew the stent graft to the vessel wall with the helical anchor axis generally parallel to the stent graft axis.

### TECHNICAL FIELD

The technical field of this disclosure is medical implantation devices, particularly, a stent graft fixation system and method of use.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include arteries such as those located within coronary, mesentery, peripheral, or cerebral vasculature; arteries; gastrointestinal tract; biliary tract; urethra; trachea; hepatic shunts; and fallopian tubes. Various types of endoluminal prostheses have also been developed with particular structures to modify the mechanics of the targeted lumen wall.

A number of vascular devices have been developed for replacing, supplementing, or excluding portions of blood vessels. These vascular devices include endoluminal vascular prostheses and stent grafts. Aneurysm exclusion devices, such as abdominal aortic aneurysm (AAA) devices, are used to exclude vascular aneurysms and provide a prosthetic lumen for the flow of blood. Vascular aneurysms are the result of abnormal dilation of a blood vessel, usually from disease or a genetic predisposition, which can weaken the arterial wall and allow it to expand. Aneurysms can occur in any blood vessel, but most occur in the aorta and peripheral arteries, with the majority of aneurysms occurring in the abdominal aorta. An abdominal aneurysm typically begins below the renal arteries and extends into one or both of the iliac arteries.

Aneurysms, especially abdominal aortic aneurysms, have been commonly treated in open surgery procedures where the diseased vessel segment is bypassed and repaired with an artificial vascular graft. While open surgery is an effective surgical technique in light of the risk of a fatal abdominal aortic aneurysm rupture, the open surgical technique suffers from a number of disadvantages. It is complex, requires a long hospital stay, requires a long recovery time, and has a high mortality rate. Less invasive devices and techniques have been developed to avoid these disadvantages. Tubular endoluminal prostheses that provide a lumen or lumens for blood flow while excluding blood flow to the aneurysm site are introduced into the blood vessel using a catheter in a less or minimally invasive technique. The tubular endoluminal prosthesis is introduced in a small diameter compressed configuration and expanded at the aneurysm. Although often referred to as stent grafts, these tubular endoluminal prostheses differ from so called covered stents in that they are not used to mechanically prop open stenosed natural blood vessels. Rather, they are used to secure graft material in a sealing engagement with the vessel wall and prop open the tubular passage through the graft without further opening the abnormally dilated natural blood vessel.

Stent grafts for use in abdominal aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or holds it in place against a vessel wall. The stent graft is secured to a vessel wall above and below the aneurysm. A proximal spring stent of the stent graft can be located above the aneurysm to provide a radial force to engage the vessel wall and seal the stent graft to the vessel wall.

One problem is that stent grafts can migrate over time after installation in the vessel. The stent graft is subject to a variety of loads, due to the force associated with the blood flowing through the stent graft, and the pulsatile blood pressure causing expansion and contraction of the arteries. Changes in the anatomy of the abdominal aortic aneurysm can contribute to the cause of migration. One attempt to prevent migration has provided the proximal spring stent with tines, barbs, hooks, and the like to puncture the vessel wall and secure the stent graft in place. Unfortunately, the wall area for prosthesis fixation above an aneurysm or other diseased vessels may be limited, making secure fixation of the prosthesis more difficult. Each hook is attached at a single point when using hooks, so the loading on the vessel wall and the hook is concentrated at the single point. Hydrodynamic loading can dislodge one or more of the hooks from the vessel wall over time and allow migration, exposing the aneurysm to blood pressure and leakage flow. The hooks are also attached to fixed positions spaced around the periphery of the stent graft, so that a poor seal and leakage occurs when the hooks are not set to the required depth.

It would be desirable to overcome the above disadvantages.

### SUMMARY OF THE INVENTION

One aspect according to the present invention provides a stent graft system for fixation to a vessel wall, the system having a helical anchor and a stent graft. The helical anchor has a number of coils and a helical anchor axis, and the stent graft has a stent graft axis. The coils are operable to sew the stent graft to the vessel wall with the helical anchor axis generally parallel to the stent graft axis.

Another aspect according to the present invention provides a system for fixing a stent graft to a vessel wall at an attachment site. The system includes an anchor guide, a driver having a driver lumen through which the anchor guide can slide, and a delivery catheter having a catheter lumen through which the driver can slide. A helical anchor is releasably connected to the driver and slidable over the anchor guide. The helical anchor is rotatable about the anchor guide to sew the stent graft to the vessel wall

Another aspect according to the present invention provides a method of fixing a stent graft to a vessel wall at an attachment site. The method includes the steps of deploying a stent graft having a stent graft lumen over the attachment site, advancing a delivery catheter having a catheter lumen into the stent graft lumen, and advancing an anchor guide through the catheter lumen until the anchor guide is adjacent to the attachment site. A helical anchor is advanced over the anchor guide to the attachment site and engaged with the vessel wall through the stent graft at the attachment site. The helical anchor is rotated to sew the stent graft to the vessel wall at the attachment site.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIGS. 1A & 1B are side and end views, respectively, of a helical anchor;

FIGS. 2A & 2B are schematic views of the distal and proximal portions of a delivery system for a helical anchor;

FIGS. 3A-3C are exploded, anchor retracted, and anchor extended schematic illustrations of a delivery system for a helical anchor;

FIGS. 4A, 4B, & 5 are schematic views of helical anchors attached to helical anchor drivers;

FIG. 6 is a side fluoroscopic view of stent graft for use with a helical anchor;

FIGS. 7A-7D are schematic views of a delivery system for a helical anchor with a curved rail positioner;

FIGS. 8A-8C are schematic views of a delivery system for a helical anchor with a spring sleeve positioner;

FIGS. 9A & 9B are schematic views of a delivery system for a helical anchor with a balloon positioner;

FIGS. 10A-10C are schematic views of a delivery system for a helical anchor with a crown ring positioner;

FIGS. 11A-11D are schematic views of deployment of a helical anchor; and

FIG. 12 is a flowchart of the steps of a method of fixing a stent graft to a vessel wall at an attachment site.

### DETAILED DESCRIPTION

Embodiments according to the invention will now be described by reference to the figures wherein like numbers refer to like structures. For the catheter, the terms “distal” and “proximal” are used herein with reference to the treating clinician: “distal” indicates an apparatus portion distant from, or a direction away from the clinician and “proximal” indicates an apparatus portion near to, or a direction towards the clinician. While for stent graft devices the proximal end is the end closest to the heart by way of blood flow path and the distal end is the end farthest from the heart by way of blood flow path.

Embodiments according to the current invention disclose devices and methods for fixation of stent grafts. While these devices and methods are described below in terms of being used to treat abdominal aortic aneurysms, it will be apparent to those skilled in the art that the devices could be used fix other devices in other vessels as well. Stent graft anchors described include helical anchors used to fix a stent graft to the vessel wall at an attachment site. The systems described include helical anchors and the delivery catheters for placing the devices at the attachment site.

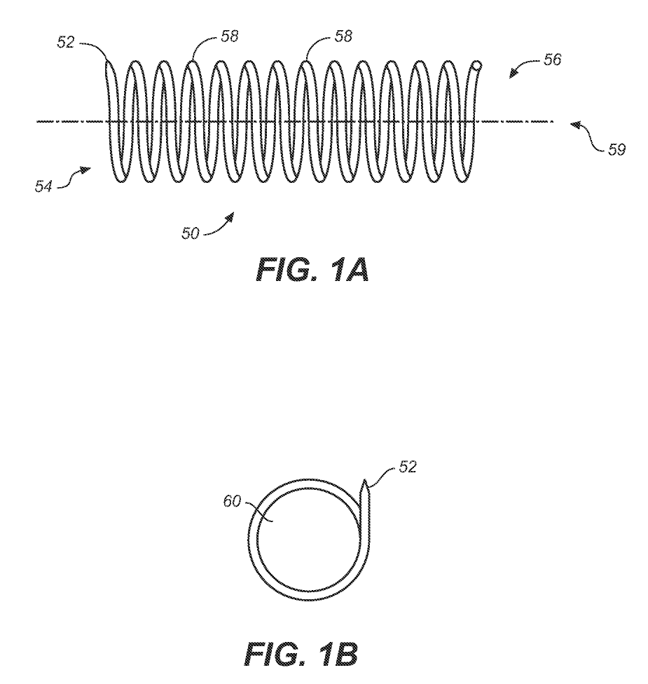

FIGS. 1A & 1B are side and end views, respectively, of a helical anchor. Helical anchor 50 is an elongated helix having a tissue penetrating sharpened tip 52 at a distal end 54 and a proximal end 56 that can be operably connected to a helical anchor driver. The helical anchor 50 includes a number of individual coils (windings) 58 along a helical anchor axis 59, which form a generally cylindrical inner channel 60 that can accommodate an anchor guide to direct deployment of the helical anchor 50. The helical anchor 50 is rotated about its axis when the sharpened tip 52 is engaged with a stent graft and vessel wall tissue to sew the helical anchor 50 to the vessel wall and fix the stent graft in position. In one configuration, the helical anchor axis 59 is generally parallel to the stent graft axis (not shown) when the stent graft is fixed in position. The diameter of the inner channel 60, the pitch of the coils 58, and/or the length of the sharpened tip 52 can be selected to provide a desired penetration depth for the helical anchor 50 in the vessel wall tissue. The wire diameter, materials, and pitch of the coils 58 can be selected to provide a desired axial flexibility for the helical anchor 50.

The helical anchor 50 can be formed of a biocompatible metallic or polymeric material having suitable resiliency. The metallic or polymeric material can be a wire coiled to make the helical anchor 50. In one embodiment, the helical anchor 50 is formed of stainless steel. In another embodiment, the helical anchor is formed of 35N LT® metal alloy wire. In yet another embodiment, the helical anchor 50 is formed of MP35N® metal alloy wire. In one embodiment, at least a portion of the helical anchor 50 is made from material having a high X-ray attenuation coefficient to enhance visibility during deployment. In one example, the helical anchor 50 is made of stainless steel wire having a diameter of 0.020 inches, with the helical anchor 50 having an inner diameter of 0.11 inches, an outer diameter of 0.150 inches, and a pitch of 12 coils per inch. The dimensions and materials of the helical anchor 50 can be selected to provide the desired performance characteristics for a desired application.

The helical anchor 50 forms an inner channel 60 to receive an anchor guide, which guides the helical anchor 50 during deployment. In one embodiment, the diameter of the inner channel 60 can be in the range of 0.10 inches to 0.20 inches, such as 0.11 inches. In one embodiment, the external diameter of the helical anchor 50 can be in the range of 0.150 inches to 0.250 inches, such as 0.150 inches.

The distance between adjacent coils 58 defines the coil pitch measured in number of coils per inch. The number of coils per inch for the helical anchor 50 can be selected for the desired degree of flexibility and resiliency. In one embodiment, the coil pitch can be in the range of 10 to 20 coils per inch, such as 12 to 14 coils per inch.

The helical anchor 50 has a generally circular shape transverse to the long axis of the helical anchor 50, and the sharpened tip 52 extends on a tangent away from the circular perimeter of the helical anchor. The sharpened tip 52 is angled away from the exterior circular perimeter of the helical anchor 50 to allow the sharpened tip 52 to penetrate vessel wall tissue when the helical anchor 50 is rotated out of a delivery catheter and in contact with an adjacent structure. The length of the sharpened tip 52 controls the depth at which the helical anchor 50 is sewn into the vessel wall tissue and depends on the diameter of the coils 58. The length of the sharpened tip 52 also controls resistance to the coil penetration. The length of the sharpened tip 52 is selected for a particular application to be long enough to assure good fixation of the helical anchor 50 to the vessel wall, but not so long that excessive force is required to rotate the helical anchor 50 when sewing the helical anchor 50 to the vessel wall.

The diameter of the metallic or polymeric wire can be selected based on design considerations, such as flexibility, delivery method, and the like. In one embodiment, the wire diameter can be in the range of 0.017 inches to 0.025 inches, such as 0.02 inches. The cross section of the wire need not be circular, but can be other shapes as desired. The wire can also include a lubricious coating, such as an MDX coating, for deliverability.

The length of the helical anchor 50 can be selected as desired for the length of the attachment region in the vessel wall available to fix the stent graft. A number of helical anchors 245 can be used with a single stent graft to assure fixation. The helical anchor 50 can have a left hand wind or a right hand wind depending on the particular application.

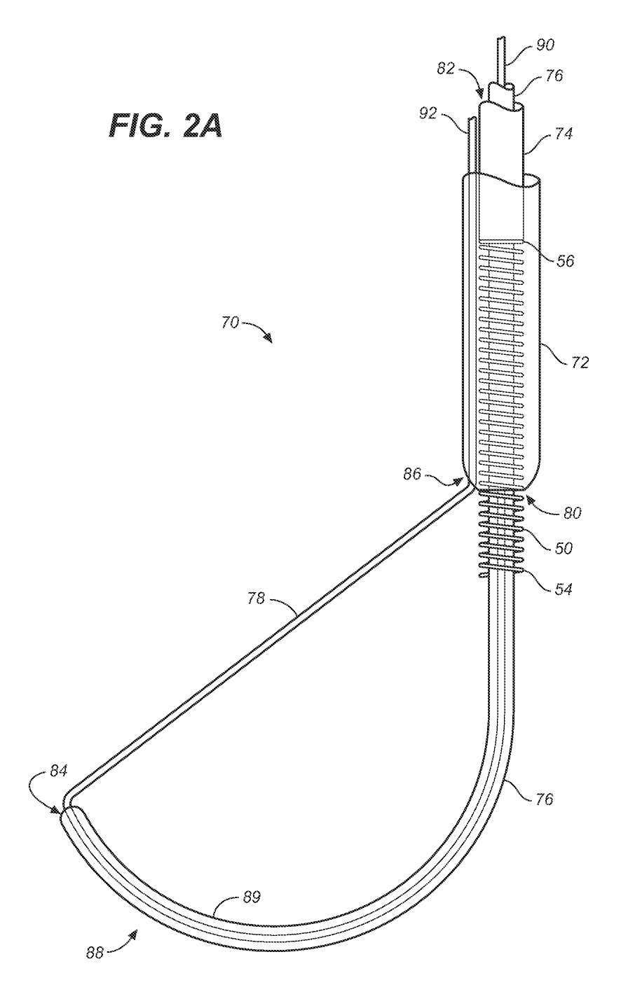

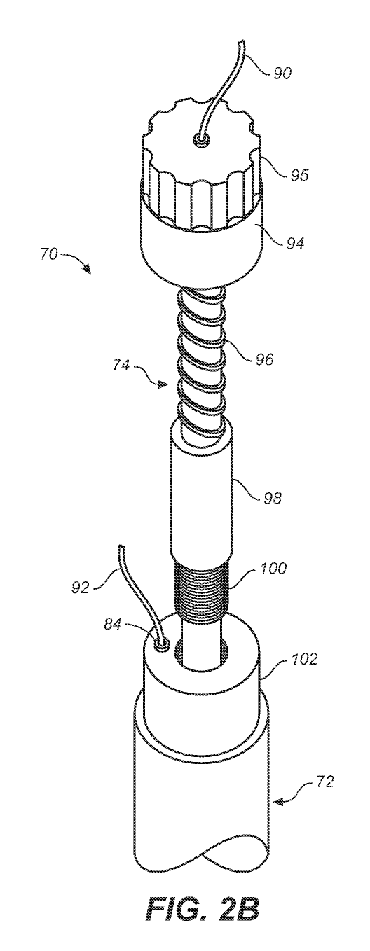

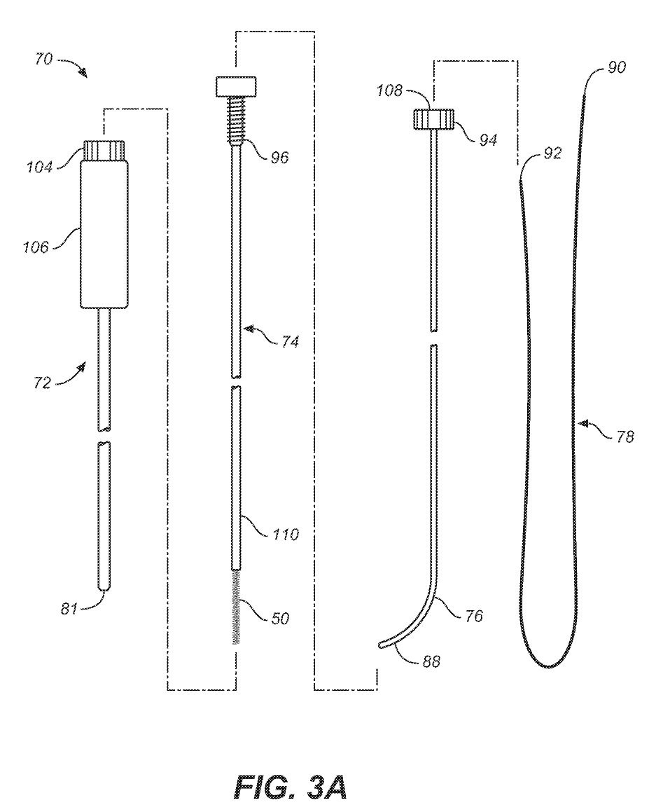

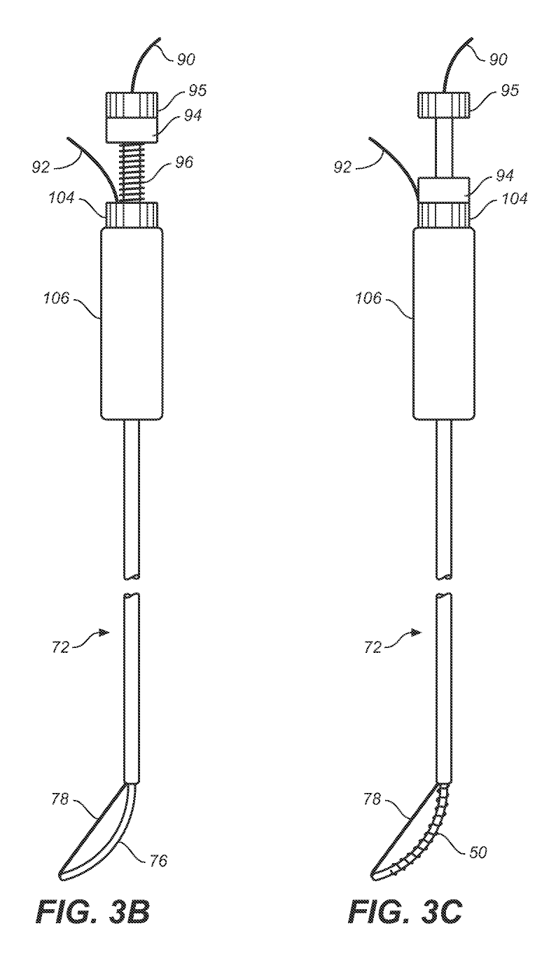

FIGS. 2A, 2B, & 3A-3C are schematic views of a delivery system for a helical anchor. In this example, the delivery system includes a curved rail as a positioner in the anchor guide to guide and aid in urging the helical anchor toward the attachment site in the vessel wall during deployment. FIGS. 2A & 2B illustrate the distal and proximal ends, respectively, of the distal and proximal portions a delivery system for a helical anchor. FIGS. 3A-3C illustrate side views of exploded, anchor retracted, and anchor extended configurations, respectively, of a delivery system for a helical anchor.

Referring to FIG. 2A, the delivery system 70 includes a delivery catheter 72, driver 74, anchor guide 76, and helical anchor 50. A positioner 88 includes a curved rail 89 at the distal end of the anchor guide 76 and tether 78. The delivery catheter 72 is a flexible elongate tube for insertion into the patient. The delivery catheter 72 includes a catheter lumen 80 for receiving the driver 74 and the anchor guide 76. The delivery catheter 72 can be made of flexible, biocompatible polymeric material such as, but not limited to, polyurethane, polyethylene, nylon, and polytetrafluoroethylene (PTFE).

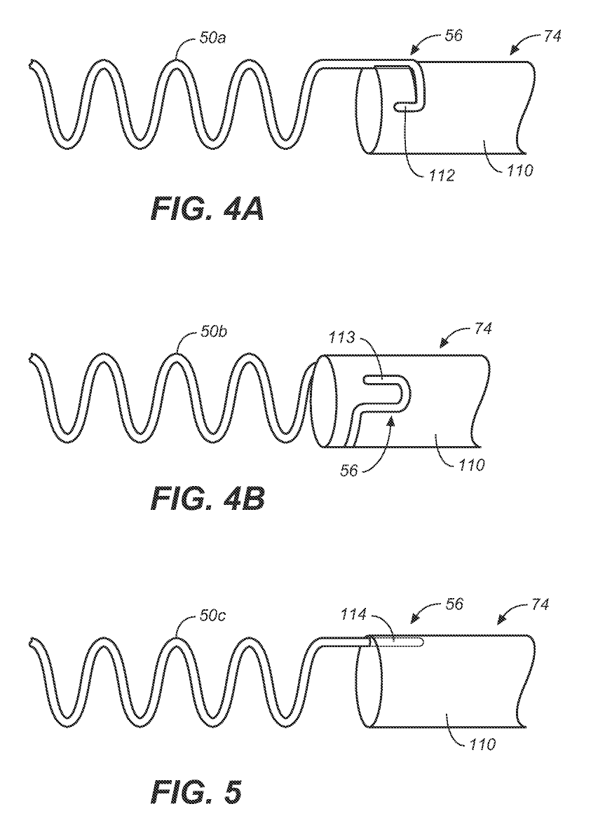

The driver 74 is an elongate tube having a distal drive end for driving the helical anchor 50. The driver 74 is able to rotate and translate longitudinally along a long axis of the catheter lumen 80 during implantation of the helical anchor 50. The distal end of the driver 74 includes a helical anchor-receiving portion for releasably holding the helical anchor 50. In one embodiment, the helical anchor-receiving portion includes a hole for receiving a pin-shaped driver portion of the proximal end of the helical anchor 50 as described for FIG. 5. In another embodiment, the helical anchor-receiving portion includes an indentation for receiving a generally U-shaped driver portion of the proximal end of the helical anchor 50 as described for FIG. 4A. In another embodiment, the helical anchor-receiving portion includes an indentation for receiving a generally wrapping driver portion of the proximal end of the helical anchor 50 as described for FIG. 4B. In one embodiment, the driver 74 includes a driver lumen 82 for receiving the anchor guide 76. The driver 74 can be made of flexible, biocompatible polymeric material such as, but not limited to, polyurethane, polyethylene, nylon, and polytetrafluoroethylene (PTFE). In one embodiment, the interior walls of the delivery catheter 72 forming the catheter lumen 80 are coated with a lubricious material such as silicone, polytetrafluroethylene (PTFE), or a hydrophilic coating. The lubricious interior walls of the delivery catheter 72 facilitate longitudinal movement of the driver 74.

The anchor guide 76 is an elongate member configured to place the helical anchor 50 at the attachment site in the vessel wall during deployment. In one embodiment, the anchor guide 76 is constructed from a material having shape memory properties so that the anchor guide 76 assumes a curved shape when the distal end of the anchor guide 76 leaves the delivery catheter 72. The anchor guide 76 can be made of a biocompatible metallic or polymeric material or combinations thereof. Fabrication of the anchor guide 76 can include chemical machining, forming, and/or heat setting of nitinol. The anchor guide 76 can include an anchor guide lumen 84 through which the tether 78 can slide.

The anchor guide 76 can have a generally semi-circular (D-shaped), circular or elliptical cross-section such that at least a portion of the exterior surface of the anchor guide 76 has a shape that is complementary to the inner circumference of the helical anchor 50. During deployment of helical anchor 50, the helical anchor 50 releasably connected to the driver 74 slides over the anchor guide 76, which guides the helical anchor 50 as it advances along the length of anchor guide 76.

During the delivery of a helical anchor 50 to an attachment site, the various components of the system are concentrically disposed within the delivery catheter 72. The arrangement of the various components within the delivery catheter 72 can be selected as desired for a particular application.

FIG. 2B illustrates the proximal end of the delivery system 70 with controls for manipulating the various components of the delivery system 70. The proximal end of the driver 74 includes an anchor driver knob 94, a threaded portion 96, and an optional lock ring 98. The lock ring 98 includes a threaded section 100 for threaded engagement with a delivery catheter ring 102. The lock ring 98 holds the threaded section 100 to the delivery catheter 72 during implantation of the helical anchor 50. In another embodiment, the lock ring 98 can be omitted and the threaded portion 96 of the driver 74 engages threads in the delivery catheter ring 102 directly. The anchor guide 76 includes a guide driver knob 95.

To deploy the helical anchor 50, the delivery system 70 is preloaded with the anchor guide 76 installed within the driver 74, the driver 74 is installed within the delivery catheter 72, and the tether 78 is threaded through the anchor guide 76 and delivery catheter 72. The lock ring 98 is screwed into the handle cap 102 with the threaded section 100. The distal tip of the anchor guide 76 and the helical anchor 50 on the driver 74 are placed at the attachment site. The driver knob 94 is turned to screw the threaded portion 96 of the driver 74 into the interior of the lock ring 98 and to sew the helical anchor 50 into the vessel wall. Once the helical anchor 50 has been implanted, the driver 74 can be disengaged from the helical anchor 50 and the delivery system 70 withdrawn from the patient. The delivery catheter 72 can be left in the patient and the procedure repeated when more than one helical anchor is to be installed.

FIGS. 3A-3C illustrate exploded, anchor retracted, and anchor extended conditions, respectively, of a delivery system for a helical anchor.

Referring to FIG. 3A, the delivery catheter 72 is an elongated generally tubular catheter having a handle 106 and a handle cap 104 at the proximal end of the delivery catheter 72. The delivery catheter 72 includes a catheter lumen (not shown) which extends the axial length of the delivery catheter 72 to distal opening 81. A helical anchor driver 74 can be disposed in the driver lumen.

The elongated helical anchor driver 74 includes a driver knob 94 on the proximal end of the driver 74 and a threaded portion 96 adjacent the driver knob 94. A distal end 110 of the driver 74 is releasably connected to a helical anchor 50. The driver 74 includes a driver lumen (not shown) through its axial length. An anchor guide 76 can be disposed in the driver lumen. The driver 74 can be made from any biocompatible material allowing the driver 74 to rotate and to move longitudinally inside of the delivery catheter 72, and carry rotational and axial load from the proximal end to the helical anchor 50.

The elongated anchor guide 76 includes a guide driver knob 95 on the proximal end of the anchor guide 76. The anchor guide 76 includes an anchor guide lumen (not shown) through its axial length. In this example, the anchor guide 76 includes a curved rail as a positioner 88 in the anchor guide 76 to guide and urge the helical anchor 50 toward the attachment site in the vessel wall during deployment. When the delivery system includes a curved rail as a positioner 88, the delivery system can also include a flexible elongated tether 78 with a first end 92 and a second end 90. The tether 78 is threaded through the anchor guide lumen and tether lumen 84 in the anchor guide 76 and the delivery catheter 72, respectively. The ends 90, 92 remain outside the patient's body during the implantation procedure. The tether 78 can be used to bow the positioner 88 and urge the helical anchor 50 toward the attachment site in the vessel wall during deployment. The delivery catheter 72, driver 74, and anchor guide 76 are flexible enough to negotiate the turns and curves required for an approach to a treatment site through a patient's vasculature.

Referring to FIG. 3B, the exploded pieces have been assembled, the driver 74 is positioned in the catheter lumen of the delivery catheter 72 and the anchor guide 76 is positioned in the driver lumen of the driver 74. In this embodiment, the threaded portion 96 of the driver 74 directly engages a complementary threaded portion (not shown) in the handle cap 104. The anchor guide 76 is advanced until the distal tip of the anchor guide 76 is at the attachment site.

Referring to FIG. 3C, the driver knob 94 is rotated so that the threaded portion 96 on the driver 74 is screwed into the complementary threaded portion of the delivery catheter 72. As the driver 74 is threaded into the delivery catheter 72, the distal portion of the driver 74 rotates and moves toward the distal opening 81 of the delivery catheter so that the distal end of the helical anchor 50 exits the delivery catheter 72 and engages targeted structures, e.g., the vessel wall. The continued rotation of the driver knob 94 continues to progressively sew the helical anchor 50 into the vessel wall. In one embodiment, contact between the driver knob 94 and the handle cap 104 acts as a stop to limit the rotation of the driver knob 94 and axial travel of the helical anchor 50. In another embodiment, the threaded portion 96 on the driver 74 is omitted and the rotation and advancement of the driver 74 within the delivery catheter 72 is controlled manually by the clinician. The distal portion of the delivery system for handling and operating can be any arrangement desired for a particular application as long as the delivery catheter 72, driver 74, and anchor guide 76 are free to slide axially relative to one another and the driver 74 is free to rotate relative to the delivery catheter 72 and anchor guide 76.

FIGS. 4A, 4B, and 5 are side views of partial portions of helical anchors attached to a helical anchor driver. FIG. 4A illustrates one embodiment of a release mechanism in which the helical anchor 50a has a generally U-shaped driver portion 112 at the proximal end 56 of the helical anchor 50a. The distal end 110 of the driver 74 includes an indentation that is sized and shaped so that the driver portion 112 at the proximal end 56 of the helical anchor 50a fits snugly into the driver 74 for delivery of the helical anchor 50a. A retractable sleeve (not shown) is disposed over the driver portion 112 and the sleeve is retracted to free the helical anchor 50a from the driver 74 once the helical anchor 50a is fully attached to the vessel wall. The driver portion, complementary indentation, and sleeve can be on the inside or outside circumference of the driver 74 as desired for a particular application. In one embodiment, the sleeve is the distal end of the delivery catheter 72.

FIG. 4B illustrates an embodiment of a release mechanism in which the helical anchor 50b has a generally wrapping driver portion 113 at the proximal end 56 of the helical anchor 50b. The distal end 110 of the driver 74 includes an indentation that is sized and shaped so that the driver portion 113 at the proximal end 56 of the helical anchor 50b fits snugly into the driver 74 for delivery of the helical anchor 50b. Part of the helical portion of the helical anchor 50b fits into the indentation as well, so that the helical portion wraps around the distal end 110 of the driver 74. A retractable sleeve (not shown) is disposed over the driver portion 113 and the sleeve is retracted to free the helical anchor 50b from the driver 74 once the helical anchor 50b is attached to the vessel wall. The driver portion, complementary indentation, and sleeve can be on the inside or outside circumference of the driver 74 as desired for a particular application. In one embodiment, the sleeve is the distal end of the delivery catheter 72.

FIG. 5 illustrates another embodiment of a release mechanism in which the helical anchor 50c has a pin-shaped driver portion in a proximal end 56 with a driver portion 114 that extends straight in a proximal direction from the helical anchor 50c. The distal end 110 of the driver 74 includes a hole for placement of the driver portion 114 of the helical anchor 50c such that the driver portion 114 fits snugly into the driver 74 during implantation. Once the helical anchor 50c is implanted, the driver 74 is retracted axially without rotation from the helical anchor 50c and the straight driver portion 114 of the proximal end 56 is pulled from the hole in the distal end 110 of the driver 74. In one embodiment, the length of the straight driver portion 114 of the helical anchor 50c can be in the range of 0.05 inches to 0.25 inches, such as 0.10 inches.

In another embodiment, the release mechanism for the helical anchor can be a fusible link between the helical anchor and the distal end of the driver. A current from a current source can be passed through the driver after the stent graft has been fixed to the attachment site to melt the fusible link. A low voltage current, such as a current driven by about 9 Volts, can pass from the proximal end of the driver, through body of the patient, to an electrode patch secured on the exterior of the patient near the attachment site. The resistance heating of the fusible link causes the fusible link to melt. The current path can include an impedance monitor to determine when the fusible link opens. The fusible link can be made of a lead-free solder, such as a solder including silver and tin.

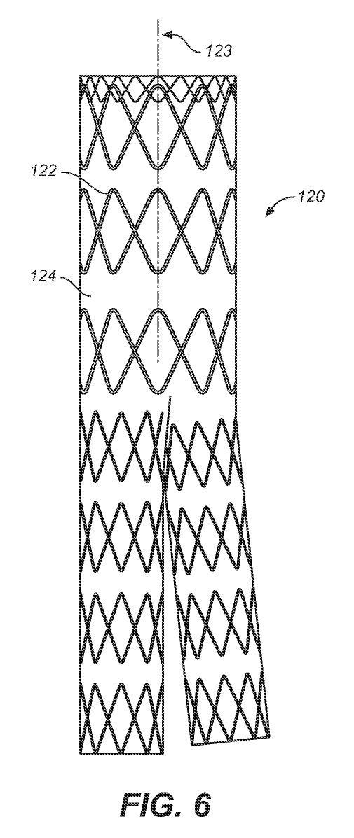

FIG. 6 is a side fluoroscopic view of stent graft for use with a helical anchor. The stent graft 120 having a stent graft axis 123 includes supports 122 to which a tubular graft material 124 is attached. The stent graft axis 123 is generally parallel to the helical anchor axis (not shown) when the stent graft is fixed in position. The stent graft 120 may be any suitable device for mechanically keeping a tubular graft open and in sealing contact with healthy surrounding tissue after being implanted at the target site. Such mechanical endoprosthetic devices, sometimes called stent grafts, are typically inserted into the target vessel, positioned across the lesion, and then expanded to bypass the weakened wall of the vessel, thereby excluding blood pressure from the aneurysm to prevent rupture of the aneurysm while the graft remains sealed to the healthy tissue after implantation of the graft. Generally, the stent graft 120 is placed from just above to just below the aneurysm in a vessel to channel flow through the stent graft and relieve the pressure from the weak aneurysm wall.

For example, the stent graft 120 may be a self-expanding or balloon expandable stent graft. Although FIG. 6 shows a bifurcated stent graft, the stent graft 120 may also be a tubular stent graft. In one embodiment, the stent graft is expanded after the stent graft is positioned across the aneurysm.

Support 122 is a support having a suitable mechanical configuration for keeping an effective blood vessel open after completion of the stent grafting procedure. For example, support 122 can be one or more stent type rings attached to graft material 124 and arranged in a manner that will allow stent graft 120 to keep the tubular graft open and in sealing contact with healthy surrounding tissue after implantation. The size and configuration of support 122 depends upon the size and configuration of the vessel to be treated. If stent type rings are used, the number and size of rings used in support 122 depends upon the size and configuration of the vessel to be treated. Individual components, such as individual rings of support 122, can be connected to each other by articulated or rigid joints or can be attached to graft material 124. The length of the stent graft 120 chosen to span the aneurysm across which it will be implanted.

Support 122 is constructed of one or more suitable implantable materials having good mechanical strength. The material can be balloon or self expanding to produce the deployed shape for the stent graft 120. For example, support 122 may be made of a suitable biocompatible metal, such as implantable quality stainless steel wire. Alternatively, support 122 is constructed of nitinol or another suitable nickel and titanium alloy. Alternatively, support 122 is constructed of any suitable metallic, plastic, or biocompatible material. The outside of the support 122 may be selectively plated with platinum, or other implantable radiopaque substances, to provide improved visibility during fluoroscopy. The cross-sectional shape of the finished support 122 may be circular, ellipsoidal, rectangular, hexagonal, square, or other polygon, depending on the size and shape of the vessel across which the system is implanted.

Stent graft material 124 is one or more suitable implantable materials having good tensile strength, such as material suitable for resisting expansion when the force associated with blood pressure is applied to it after completion of the stent grafting procedure. For example, graft material 124 is a suitable biocompatible plastic, such as implantable quality woven polyester. In some embodiments, graft material 124 includes components made of collagen, albumin, an absorbable polymer, or biocompatible fiber. Alternatively, graft material 124 is one or more suitable metallic, plastic, or non-biodegradable materials.

The size and configuration of graft material 124 depends upon the size and configuration of the aneurysm to be treated and is selected to generally match the size of support 122 to which it is attached. According to one embodiment, graft material 124 is formed of one unitary woven polyester tube.

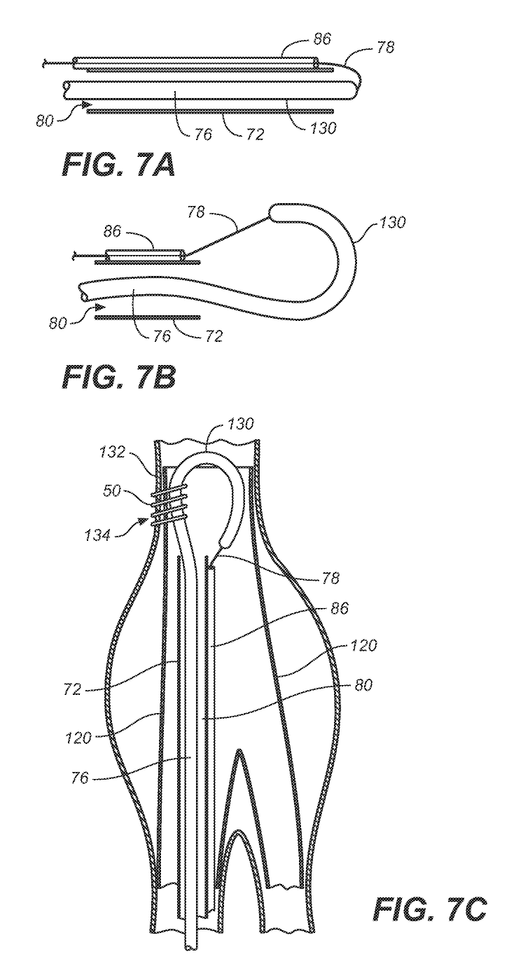

FIGS. 7A-10C illustrate embodiments of delivery systems for helical anchors. Each of the embodiments includes a positioner to urge the helical anchor toward the attachment site in the vessel wall during deployment. The positioners shown include a curved rail, a spring, a balloon, and a crown ring.

FIGS. 7A-7D are schematic views of a delivery system for a helical anchor using a curved rail positioner. FIG. 7A illustrates the curved rail positioner 130 within the catheter lumen 80 of the delivery catheter 72. In this embodiment, the curved rail positioner 130 is the distal end of the anchor guide 76. The tip of the curved rail positioner 130 is operably connected to a tether 78, which passes through a tether lumen 86 in the delivery catheter 72 to the exterior of the patient. In one embodiment, the anchor guide 76 includes an anchor guide lumen (not shown) along the axial length and the tether 78 continues through the anchor guide lumen to the exterior of the patient. FIG. 7B illustrates the curved rail positioner 130 extending from the catheter lumen 80 of the delivery catheter 72. The curved rail positioner 130 is part of the anchor guide 76, made of a biocompatible metallic or polymeric material or combinations thereof, and can be preformed into a curve or made of shape memory material that assumes a curve on exiting the catheter lumen 80. The curved rail positioner 130 is extended from the catheter lumen 80 by pushing the anchor guide 76 into the catheter lumen 80. FIG. 7C illustrates the curved rail positioner 130 extended from the catheter lumen 80 of the delivery catheter 72 with the helical anchor 50 deployed and sewn through the stent graft 120 into the vessel wall 132. The combination of pushing the anchor guide 76 in the catheter lumen 80 and pulling the tether 78 in the tether lumen 86 flexes the curved rail positioner 130 into the wall of the stent graft 120 across the stent graft 120 from the attachment site 134 to urge the helical anchor 50 on the anchor guide 76 toward the attachment site 134 in the vessel wall 132. The helical anchor 50 is delivered to the attachment site 134 and sewn into the vessel wall 132 by the driver (not shown).

FIG. 7D is a schematic sectional view of a guide rail and surrounding helical anchor. In this embodiment, the guide rail 134a is D-shaped (semicircular—though a rectangle with two rounded corners is shown in FIG. 2D), with the radiused portion of the D-shape contacting the inner circumference of the helical anchor 50. In this example, the guide rail 134a includes a core 137. The guide rail 134a urges the helical anchor 50 to a position close to the graft material so that as the helical anchor is rotated it moves around the stent graft when sewing the helical anchor 50 to the graft material 124 and vessel wall. The stiffness of the guide rail is selected to complement the stiffness of the helical anchor, so the helical anchor follows the guide rail during helical anchor attachment. A semi-circular D-shape guide rail is sized to have a cross sectional area that is approximately 25% of the cross sectional area of the approximately circular inner diameter of the helical anchor (FIG. 7D is not to scale).

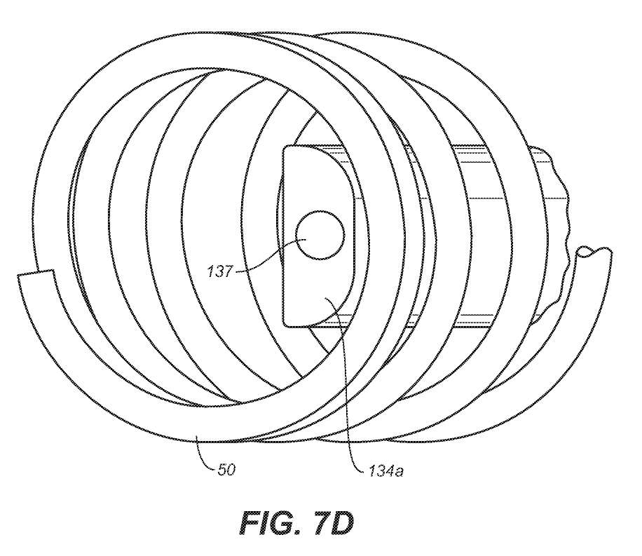

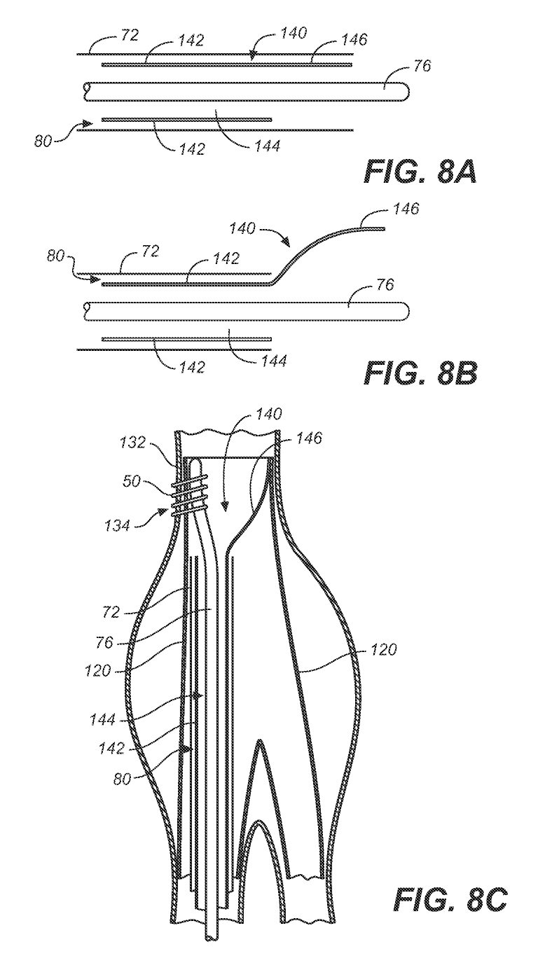

FIGS. 8A-8C are schematic views of a delivery system for a helical anchor using as a spring sleeve positioner. FIG. 8A illustrates the spring sleeve positioner 140 within the catheter lumen 80 of the delivery catheter 72. In this embodiment, the spring sleeve positioner 140 includes a sleeve 142 having a sleeve lumen 144 and a spring arm 146 at the distal end of the sleeve 142. The spring sleeve positioner 140 is slidably disposed within the catheter lumen 80 of the delivery catheter 72, and the anchor guide 76 is slidably disposed within the sleeve lumen 144. FIG. 8B illustrates the spring sleeve positioner 140 extending from the catheter lumen 80 of the delivery catheter 72. The spring sleeve positioner 140 can be preformed into a curve or made of shape memory material that assumes a curve on exiting the catheter lumen 80. The spring sleeve positioner 140 is extended from the catheter lumen 80 by pushing the sleeve 142 into the catheter lumen 80. FIG. 8C illustrates the spring sleeve positioner 140 extended from the catheter lumen 80 of the delivery catheter 72 with the helical anchor 50 deployed and sewn through the stent graft 120 into the vessel wall 132. The force of the spring arm 146 on the wall of the stent graft 120 across the stent graft 120 from the attachment site 134 urges the helical anchor 50 toward the attachment site 134 in the vessel wall 132 through the force on the sleeve 142, the delivery catheter 72, and the anchor guide 76. The helical anchor 50 is delivered to the attachment site 134 and sewn into the vessel wall 132 by the driver (not shown).

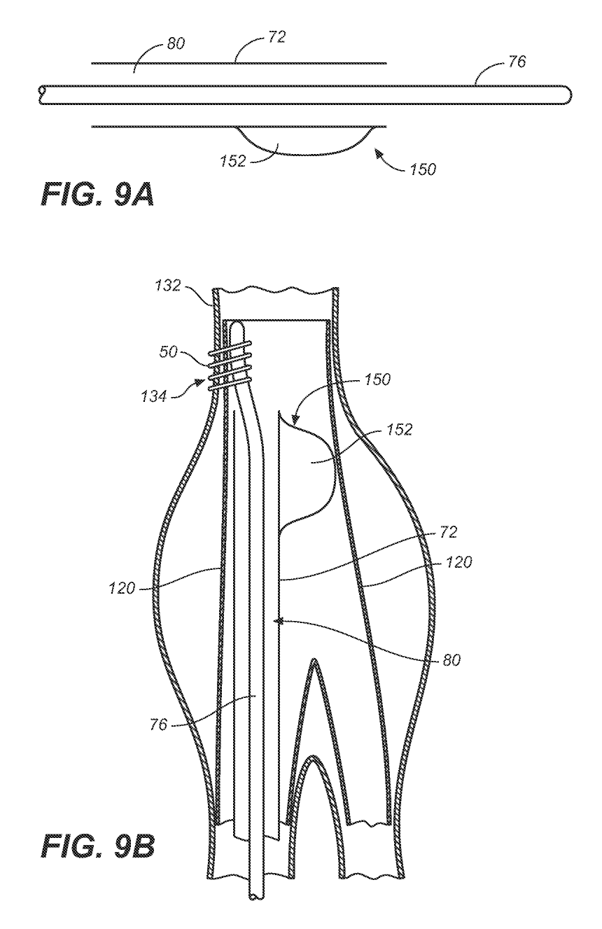

FIGS. 9A & 9B are schematic views of a delivery system for a helical anchor using a balloon positioner. FIG. 9A illustrates the balloon positioner 150 with a balloon 152 deflated. In this embodiment, the balloon positioner 150 is the balloon 152 connected to the exterior distal part of the delivery catheter 72. The delivery catheter 72 delivers the balloon positioner 150 to the attachment site 134 with the balloon 152 deflated. The balloon 152 is connected to external fluid sources so that the balloon 152 can be inflated and deflated.

FIG. 9B illustrates the balloon positioner 150 with the balloon 152 inflated, and the helical anchor 50 deployed and sewn through the stent graft 120 into the vessel wall 132. The anchor guide 76 can include a preformed shape or be made of shape memory material that assumes a predetermined shape on exiting the catheter lumen 80. The pressure of the inflated balloon 152 on the wall of the stent graft 120 across the stent graft 120 from the attachment site 134 urges the helical anchor 50 toward the attachment site 134 in the vessel wall 132 through the force on the delivery catheter 72 and the anchor guide 76. The helical anchor 50 is delivered to the attachment site 134 and sewn into the vessel wall 132 by the driver (not shown).

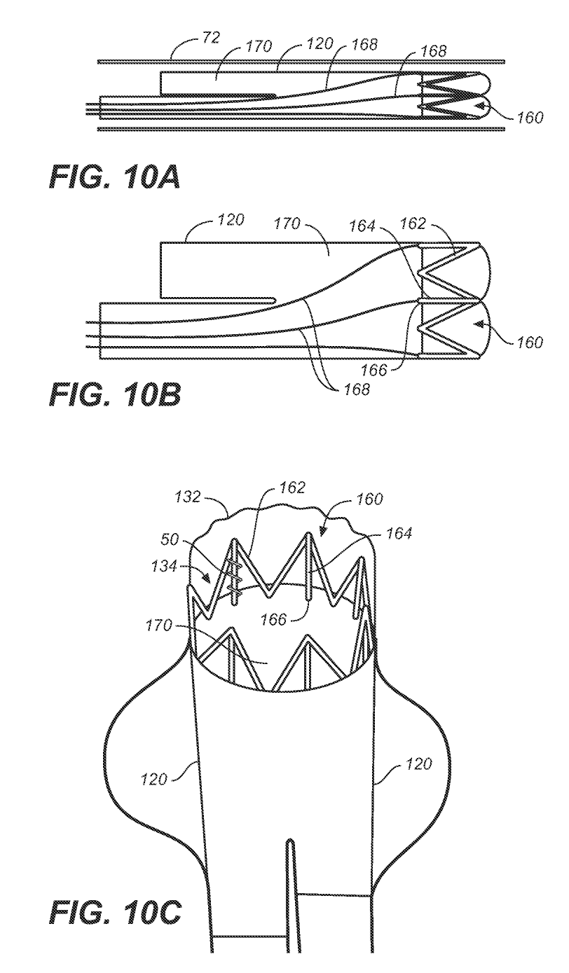

FIGS. 10A-10C are schematic views of a delivery system for a helical anchor using a crown ring positioner. FIG. 10A illustrates a stent graft 120 with crown ring positioner 160 compressed within the catheter lumen 80 of the delivery catheter 72. The stent graft 120 is delivered to the deployment site through the delivery catheter 72, and can be expanded with a balloon or can be self-expanding. FIG. 10B illustrates stent graft 120 and crown ring positioner 160 in an expanded configuration. In this embodiment, the crown ring positioner 160 includes a sinusoidal ring 162 with anchor posts 164 attached to the peaks of the sinusoidal ring 162. The free end 166 of each anchor post 164 away from the end attached to the peak of the sinusoidal ring 162 is free to allow the helical anchor 50 to slide over the anchor post 164. The free end 166 of each anchor post 164 is attached to a guide tether 168 through the stent graft lumen 170 that the driver (not shown) can follow to the attachment site 134. The number of helical anchors installed around the sinusoidal ring 162 can vary as desired for a particular application, so that each of the peaks of the sinusoidal ring 162 need not have an anchor post. FIG. 10C illustrates the stent graft 120 deployed in the vessel with the helical anchor 50 deployed and sewn through the stent graft 120 into the vessel wall 132. The force of the crown ring positioner 160 against the vessel wall across the stent graft 120 from the attachment site 134 urges the helical anchor 50 on the anchor post 164 toward the attachment site 134 in the vessel wall 132. An anchor guide (not shown) can be disposed over and follow the guide tether 168 to the attachment site 134. A driver (not shown) with a helical anchor 50 on its distal end can be disposed over the anchor guide and deliver the helical anchor 50 on its distal end to the attachment site 134. The driver sews the helical anchor 50 disposed around the anchor post 164 into the vessel wall 132. After the helical anchor 50 is deployed, the guide tether 168 can be clipped off with a cutter (not shown) that is part of the distal end of the driver or anchor guide and the guide tether removed.

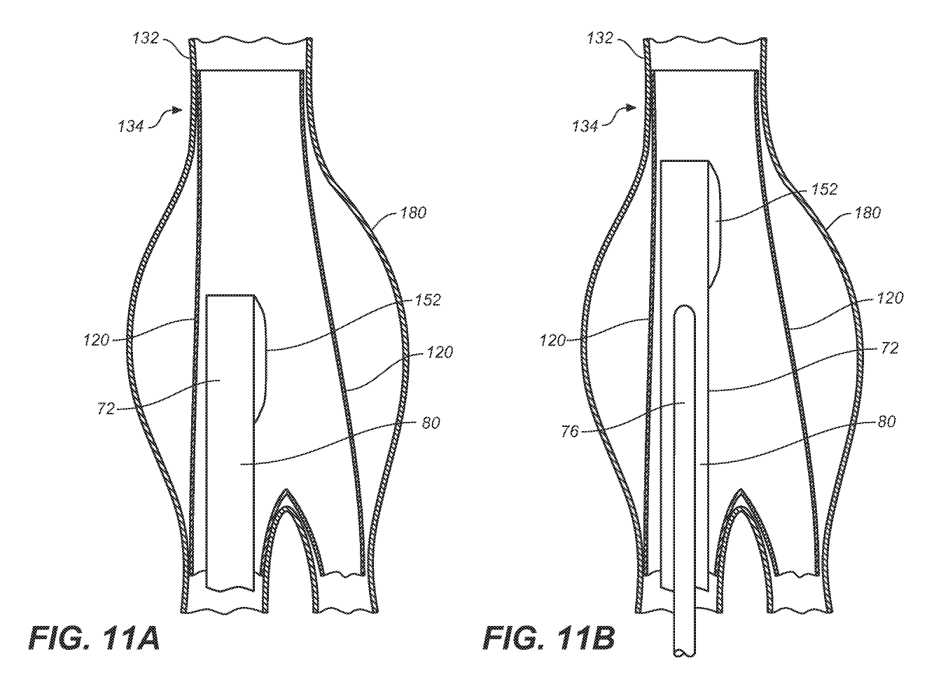

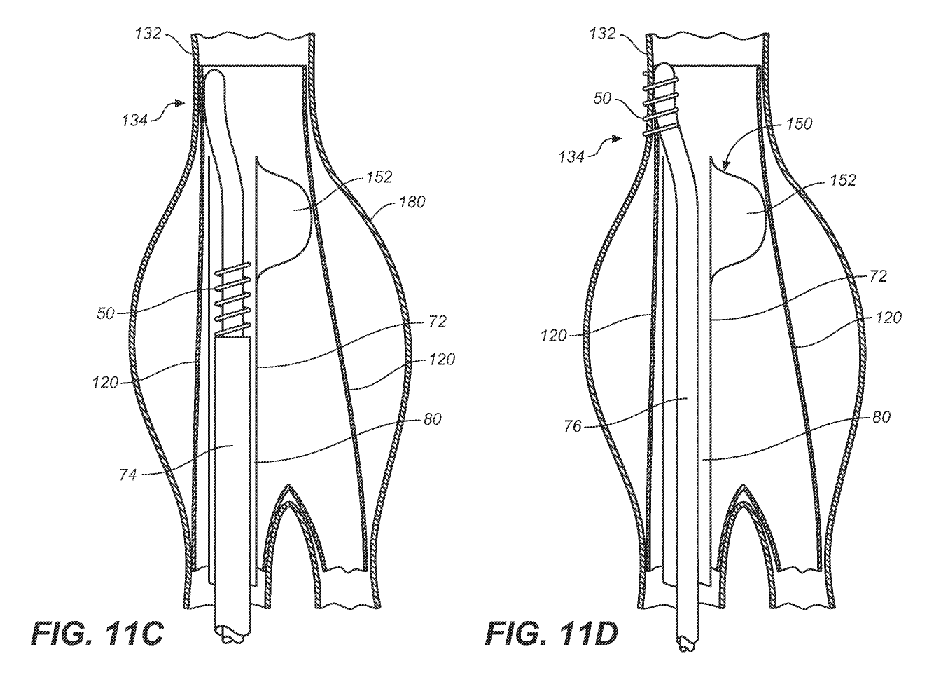

FIGS. 11A-11D are schematic views of deployment of a helical anchor. In this example, the positioner is a balloon positioner 150. Referring to FIG. 11A, the stent graft 120 has been deployed in an aneurysm 180. The delivery catheter 72 advances into the stent graft 120 and delivers the balloon positioner 150 to the attachment site 134 with the balloon 152 deflated. In this example, the balloon positioner 150 is the balloon 152 connected to the exterior distal part of the delivery catheter 72. Referring to FIG. 11B, the distal end of the delivery catheter 72 has been positioned proximally to the attachment site 134. An anchor guide 76 is advanced toward the attachment site 134 through the catheter lumen 80. Referring to FIG. 11C, the distal end of the anchor guide 76 has been positioned adjacent to the attachment site 134 and the balloon 152 inflated. A helical anchor 50 releasably connected to a driver 74 is advanced toward the attachment site 134 through the catheter lumen 80. The pressure of the inflated balloon 152 on the wall of the stent graft 120 across the stent graft 120 from the attachment site 134 urges the helical anchor 50 toward the attachment site 134 in the vessel wall 132 through the force on the delivery catheter 72 and the anchor guide 76. Referring to FIG. 11D, the helical anchor 50 has been sewn into the vessel wall 132 by the driver 74, which has been detached from the helical anchor 50 and withdrawn. The helical anchor axis is generally parallel to the stent graft axis. The balloon 152 can be deflated, and the anchor guide 76 and delivery catheter 72 withdrawn. The anchor guide 76 and delivery catheter 72 can be used to deploy a number of helical anchors before they are withdrawn.

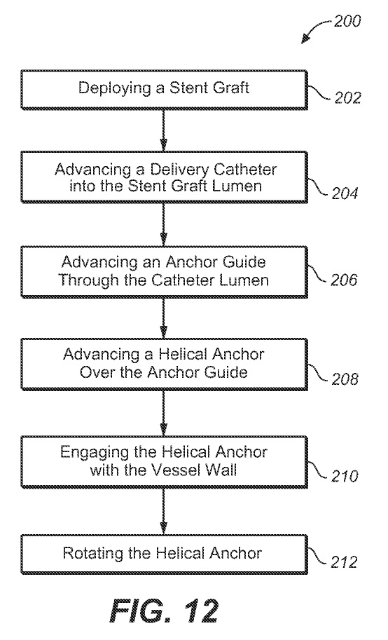

FIG. 12 is a flowchart of the steps of a method of fixing a stent graft to a vessel wall at an attachment site. The method 200 includes deploying a stent graft 202 over the attachment site, the stent graft having a stent graft lumen; advancing a delivery catheter into the stent graft lumen 204, the delivery catheter having a catheter lumen; advancing an anchor guide through the catheter lumen 206 until the anchor guide is adjacent to the attachment site; advancing a helical anchor over the anchor guide 208 to the attachment site; engaging the helical anchor with the vessel wall 210 through the stent graft at the attachment site; and rotating the helical anchor 212 to sew the stent graft to the vessel wall at the attachment site

The method 200 can further include urging the helical anchor toward the attachment site. The urging can be accomplished by flexing a curved rail positioner, extending a spring sleeve positioner, inflating a balloon positioner, or expanding a crown ring positioner, as appropriate for the type of positioner provided for a particular application.

While specific embodiments according to the invention are disclosed herein, various changes and modifications can be made without departing from its spirit and scope.

1. A stent graft system for fixation to a vessel wall comprising:
a helical anchor having a plurality of coils and a helical anchor axis; and
a stent graft having a stent graft axis;
wherein the plurality of coils are operable to sew the stent graft to the vessel wall with the helical anchor axis generally parallel to the stent graft axis.

2. The stent graft system of claim 1 wherein the helical anchor further comprises a sharpened tip at an end of the helical anchor.

3. The stent graft system of claim 1 wherein the stent graft further comprises a crown ring connected to one end of the stent graft, the crown ring having a sinusoidal ring and at least one anchor post, wherein one end of the at least one anchor post is attached to the sinusoidal ring and another end of the at least one anchor post is a free end, the helical anchor being disposed about the at least one anchor post.

4. The stent graft system of claim 3 further comprising a guide tether attached to the free end.

5. A system for fixing a stent graft to a vessel wall at an attachment site, the system comprising:
an anchor guide;
a driver having a driver lumen through which the anchor guide can slide;
a delivery catheter having a catheter lumen through which the driver can slide; and
a helical anchor releasably connected to the driver and slidable over the anchor guide;
wherein the helical anchor is rotatable about the anchor guide to sew the stent graft to the vessel wall.

6. The system of claim 5 further comprising a positioner operable to urge the helical anchor toward the attachment site.

7. The system of claim 6 wherein the delivery catheter defines a tether lumen, and the positioner is a curved rail positioner comprising:
a curved rail at a distal end of the anchor guide; and
a tether slidably disposed in the tether lumen and attached to the distal end.

8. The system of claim 6 wherein the delivery catheter defines a tether lumen, the anchor guide defines an anchor guide lumen, and the positioner is a curved rail positioner comprising:
a curved rail at a distal end of the anchor guide; and
a tether slidably disposed in the tether lumen and the anchor guide lumen.

9. The system of claim 6 wherein the positioner is a spring sleeve positioner comprising:
a sleeve having a sleeve lumen; and
a spring arm connected to a distal end of the sleeve;
wherein the anchor guide is slidably disposed in the sleeve lumen and the sleeve is slidably disposed in the catheter lumen.

10. The system of claim 6 wherein the positioner is a balloon positioner comprising a balloon connected to an exterior distal part of the delivery catheter.

11. The system of claim 6 wherein the positioner is a crown ring positioner comprising a crown ring connected to one end of the stent graft, the crown ring having a sinusoidal ring and at least one anchor post, wherein one end of the at least one anchor post is attached to the sinusoidal ring and the other end of the at least one anchor post is a free end.

12. The system of claim 11 further comprising a guide tether attached to the free end.

13. The system of claim 5 wherein the helical anchor further comprises a sharpened tip at an end of the helical anchor.

14. The system of claim 5 wherein the helical anchor is releasably connected to the driver with a generally U-shaped driver portion at a proximal end of the helical anchor engaged in an indentation in the driver.

15. The system of claim 5 wherein the helical anchor is releasably connected to the driver with a generally wrapping driver portion at a proximal end of the helical anchor engaged in an indentation in the driver.

16. The system of claim 5 wherein the helical anchor is releasably connected to the driver with a pin-shaped driver portion at a proximal end of the helical anchor engaged in a hole in the driver.

17. The system of claim 5 wherein the helical anchor is releasably connected to the driver with a fusible link.

18. A method of fixing a stent graft to a vessel wall at an attachment site, the method comprising:
deploying a stent graft over the attachment site, the stent graft having a stent graft lumen;
advancing a delivery catheter into the stent graft lumen, the delivery catheter having a catheter lumen;
advancing an anchor guide through the catheter lumen until the anchor guide is adjacent to the attachment site;
advancing a helical anchor over the anchor guide to the attachment site;
engaging the helical anchor with the vessel wall through the stent graft at the attachment site; and
rotating the helical anchor to sew the stent graft to the vessel wall at the attachment site.

19. The method of claim 18 further comprising urging the helical anchor toward the attachment site.

20. The method of claim 19 wherein the urging comprises flexing a curved rail positioner.

21. The method of claim 19 wherein the urging comprises extending a spring sleeve positioner.

22. The method of claim 19 wherein the urging comprises inflating a balloon positioner.

23. The method of claim 19 wherein the urging comprises expanding a crown ring positioner.
